(19) United States
(12) Patent Application Publication
(10) Pub. No.: US 2008/0119744 A1
(43) Pub. Date: May 22, 2008

(54) Cuff structure for sphygmomanometers

(75) Inventors: Paul Yang, Taipei (TW); Shan-Yi Yu, Taipei (TW); Yi-Tsung Lin, Taipei (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, Chung Ho City (TW)

(21) Appl. No.: 11/600,853

(22) Filed: Nov. 17, 2006

(51) Int. Cl. A61B 5/022

(52) U.S. Cl. 600/490

(57) ABSTRACT

A cuff structure for sphygmomanometers includes a cuff body wrapped around an airbag, and first, second and third adhesive latching surfaces arranged sequentially on the cuff body. The first adhesive latching surface is made of a single material; the second adhesive latching surface is composed of first and second adhesive latching portions made of different materials; one of the adhesive latching portions is made of the same material of the first adhesive latching surface; and an adhesive latching portion of the third adhesive latching surface is adhered with an adhesive latching portion of the second adhesive latching surface, such that the cuff body can be wrapped around a user's arm. When the second and third adhesive latching surfaces are folded towards the first adhesive latching surface, the adhesive latching portions made of different adjacent materials are adhered closely to prevent the airbag from getting loosened when the airbag is inflated.

### BACKGROUND OF THE INVENTION

1. Field of the Invention

The present invention relates to an improved cuff structure for sphygmomanometers, and more particularly to an adhesive latching portion made of different adjacent materials and the adhesive latching portion works together with a cuff body wrapped around a user's arm, such that the cuff body can be adjusted around the diameter of the user's arm to wrap the cuff body appropriately and tightly around the user's arm. In the processes of measuring a blood pressure and inflating and pressurizing the airbag, the cuff body will not get loosened easily, so as to measure blood pressures successfully.

2. Description of the Related Art

Referring to FIGS. 1 and 2 for wrapping an airbag of a traditional sphygmomanometer, the sphygmomanometer includes a multi-channel tube, and an end of the multi-channel tube is connected to a pressurizing apparatus (not shown in the figure), and another end is connected to an airbag 100 wrapped at a place of a human arm or wrist, and the surface of the airbag 100 has a cuff 200, and an end of the cuff 200 has a retaining ring 201 for passing another end of the cuff 200 and adjusting the wrap around the user's arm according to the diameter of the user's arm (as shown in FIG. 1), and the surface of the cuff 200 sequentially has first and second adhesive latching surfaces 210, 211, and the first adhesive latching surface 210 is made of a material different from that of the second adhesive latching surface 211, and the first and second adhesive latching surfaces 210, 211 are adhered and attached with each other. The airbag 100 is divided into a regular type and a super large type to fit different diameters of arms, wherein the regular type (that fits an arm with a diameter of 9-13 inches) is suitable for the arms of Orientals and the standard type of build of Europeans or Americans, and the super large type (that fits an arm with a diameter of 13-17 inches) is used mostly for the arms of fat people. Taking the production cost into consideration, manufacturers produce a one-size-fits-all airbag (which is a super large airbag) 100 to fit different diameters of consumers' arms, so that a user can adjust the cuff 200 wrapped on a user's arm according to the diameter of the user's arm when measuring a blood pressure. Referring to FIG. 2, the cuff 200 will be tangled if the diameter of the user's arm is of a regular size, and thus a portion of a first adhesive latching surface 210 is passed through a retaining ring 201 in a folded form, and the first adhesive latching surface 210 is folded and attached onto the first adhesive latching surface 210 made of the same material. If the diameter of the arm is of a super large, the cuff 200 will be tangled, and a portion of a second adhesive latching surface 211 will not be passed through the retaining ring 201, and the second adhesive latching surface 211 will be folded and attached on the second adhesive latching surface 211 made of the same material. As a result, the adhesion is insecure, so that the cuff 200 will get loosened easily and the blood pressure measurement will be inaccurate, when the airbag is inflated for measuring a blood pressure.

### SUMMARY OF THE INVENTION

In view of the shortcomings of the airbag of a traditional sphygmomanometer wrapped around arms of different diameters that cannot be thoroughly and tightly attached on the arms, and the airbag getting loosened and resulting in an inaccurate blood pressure measurement when the airbag is inflated, the inventor of the present invention based on years of experience to conduct extensive experiments and modifications, and finally developed an improved cuff structure for sphygmomanometers in accordance with the present invention.

Therefore, it is a primary objective of the present invention to provide an improved cuff structure for sphygmomanometers that includes an airbag, a cuff body disposed on a surface of the airbag, and first, second and third adhesive latching surfaces arranged sequentially on the cuff body, wherein the first adhesive latching surface is made of a single material, and the second adhesive latching surface is composed of an adhesive latching portion made of different adjacent materials and adhered with adjacent adhesive latching portions, and one of the adhesive latching portions is made of the same material as the first adhesive latching surface, and an adhesive latching portion of the third adhesive latching surface is arranged alternately and adhered with an adhesive latching portion of the second adhesive latching surface, such that when a blood pressure is measured, the cuff body is wrapped around a user's arm and passed through a retaining ring, and a user can adjust the cuff body to fit the diameter and size of the user's arm. When the second and third adhesive latching surfaces are folded towards the first adhesive latching surface, the adhesive latching portions made of different adjacent materials are adhered and attached tightly and wrapped around the periphery of a user's arm to fit both regular and super large arms. When the airbag is pressurized, the airbag will not get loosened easily in order to complete a blood pressure measurement successfully. The foregoing first, second and third adhesive latching surfaces could be made of Velcro.

To make it easier for our examiner to understand the objective, shape, assembly, structure, characteristics and performance of the present invention, the following embodiments accompanied with the related drawings are described in details.

### BRIEF DESCRIPTION OF THE DRAWINGS

FIG. 1 is a schematic view of a traditional apparatus;

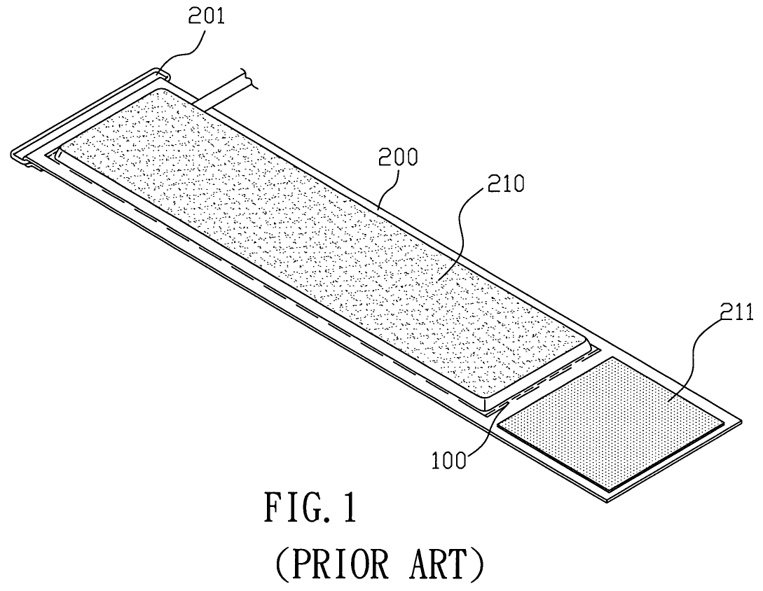

FIG. 2 is a schematic view of an application of a traditional apparatus;

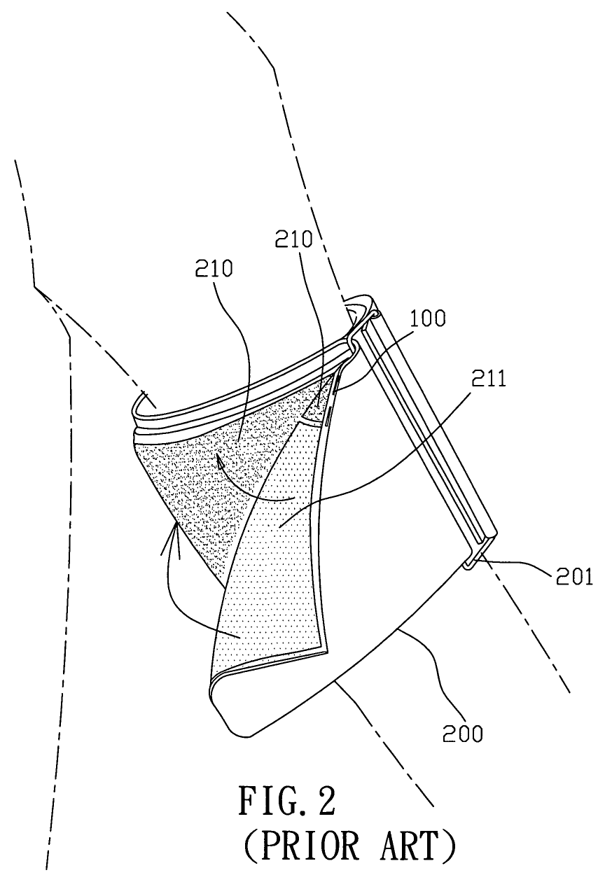

FIG. 3 is a perspective view of the present invention;

FIG. 4 is a schematic view of wearing a cuff on an arm of a regular size in accordance with the present invention; and

FIG. 5 is a schematic view of wearing a cuff on an arm of a super large size in accordance with the present invention.

### DETAILED DESCRIPTION OF THE PREFERRED EMBODIMENTS

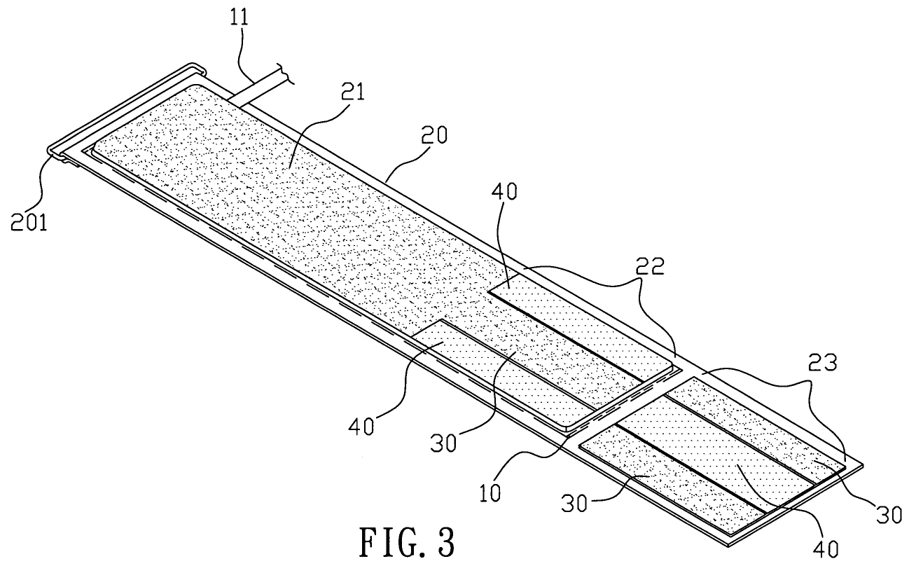

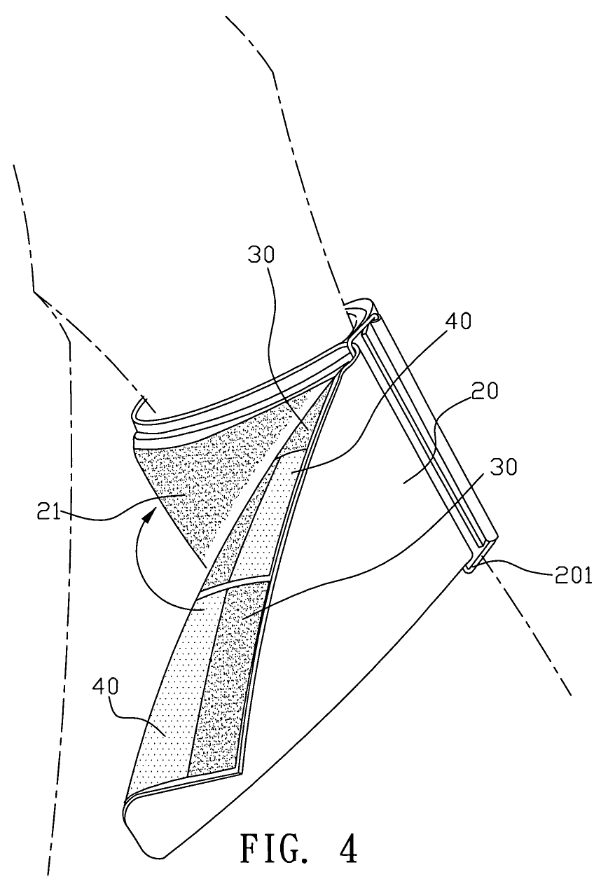

Referring to FIGS. 3 and 4 for an improved cuff structure for sphygmomanometers of the present invention, the cuff structure comprises an inflatable airbag 10, a cuff body 20 covered on a surface of the airbag 10 for wrapping an examinee's arm, a retaining ring 201 installed at an end of the cuff body 20 for passing through another end of the cuff body 20 and folded onto the cuff body 20 for adjusting the cuff body 20 to fit different sizes of arms, and a connector 11 disposed at a predetermined position and coupled to a pressurizing apparatus (not shown in the figure) of the sphygmomanometer.

The surface of the cuff body 20 sequentially includes a first, a second and a third adhesive latching surfaces 21, 22, 23 which are adhesive tapes in this preferred embodiment, wherein the first adhesive latching surface 21 is composed of a single material, and the second adhesive latching surface 22 has first and second adhesive latching portions 30, 40 made of different adjacent materials and adhered with each other, and one of the adhesive latching portions is made of the same material of the first adhesive latching surface 21, and the first adhesive latching portion 30 and the first adhesive latching surface 21 are made of the same material, and the third adhesive latching surface 23 has first and second adhesive latching portions 30, 40 made of the same material of the second adhesive latching surface 22. However, the first and second adhesive latching portions 30, 40 on the third adhesive latching surface 23 are arranged alternately with second and first adhesive latching portions 40, 30 on the second adhesive latching surface 22 and adhered with each other.

Referring to FIG. 4 for a schematic view of wearing a cuff body 20 on a user's arm of a regular size, an end of the cuff body 20 is passed through a retaining ring 201 and folded towards a first adhesive latching surface 21, and thus second and third adhesive latching surfaces 22, 23 and a portion of the first adhesive latching surface 21 are passed through the retaining ring 201 and folded. Although a portion of the folded first adhesive latching surface 21 and the unfolded first adhesive latching surface 21 are not adhered or attached with each other due to the different materials, the first adhesive latching surface 21 of the second and third adhesive latching surfaces 22, 23 can be adhered and latched tightly with the second adhesive latching portion 40 made of a different material. Therefore, the airbag 10 will not get loosened easily during the process of inflating airbag 10, and the blood pressure can be measured successfully. The foregoing first, second and third adhesive latching surfaces could be made of Velcro.

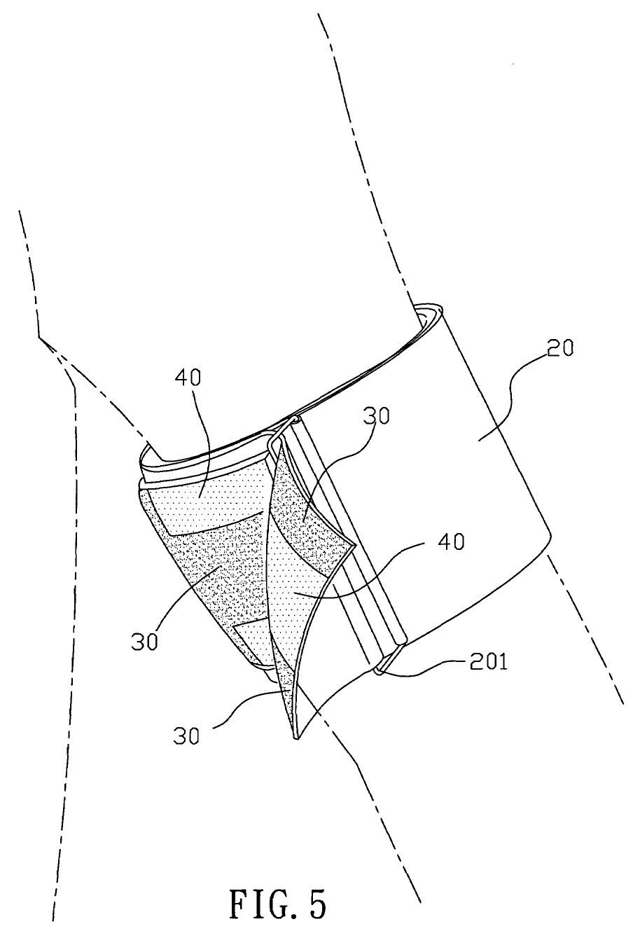

Referring to FIG. 5 for a schematic view of wearing a cuff body 20 on a user's arm of a regular size, the cuff body 20 is wrapped around a user's arm of a super large size and an end of the cuff body 20 is passed through a retaining ring 201 and folded towards a first adhesive latching surface 21 (not shown in the figure), such that only a third adhesive latching surface 23 will be folded (the size of folded portion varies with the diameter of the user's arm, and this embodiment shows one of the cases) as the third adhesive latching surface 23 is passed through the retaining ring 201. Since first and second adhesive latching portions 30, 40 folded onto the third adhesive latching surface 23 are arranged alternately and adhered tightly with first and second adhesive latching portions 30, 40 on the second adhesive latching surface 22, therefore the airbag 10 (not shown in the figure) will not get loosened easily during the process of inflating the airbag 10, so as to measure blood pressures successfully.

When a user measures a blood pressure, the user wraps the cuff body 20 around the user's arm and adjusts the cuff body 20 according to the diameter of the user's arm. When the cuff body 20 is folded, the first and second adhesive latching portions 30, 40 arranged alternately on the second and third adhesive latching surfaces 22, 23 are adhered and wrapped tightly around the periphery of the user's arm. The foregoing first, second and third adhesive latching surfaces could be made of Velcro.

In summation of the above description, the cuff structure for sphygmomanometers in accordance with the present invention herein provides a simple and easy structure, saves the cost, enhances the performance, overcomes the shortcomings of the prior art, and further complies with the patent application requirements, and thus the invention is duly filed for patent application.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

1. An improved cuff structure for sphygmomanometers, comprising:
an airbag, used for inflating;
a cuff body, covered on a surface of said airbag, configured to be wrapped about a user's arm, and on one side sequentially having a first, a second and a third adhesive latching surfaces;
said first adhesive latching surface, being composed of a single material;
said second adhesive latching surface, with an adhesive latching surface having first and second adhesive latching portions composed of different adjacent materials, and said first and second adhesive latching portions being adhered and latched with each other, and one of said adhesive latching portions of said first and second adhesive latching portions being made of the same material of said first adhesive latching surface; and
said third adhesive latching surface, with an adhesive latching surface having first and second adhesive latching portions arranged alternately with said adhesive latching portions of said second adhesive latching surface and adhered and latched with each other.

2. The improved cuff structure for sphygmomanometers of claim 1, wherein said cuff body includes a retaining ring for adjusting the area of wrapping said cuff body around an arm.

3. The improved cuff structure for sphygmomanometers of claim 1, wherein said first, second and third adhesive latching surfaces are made of a hook and loop fastener.
